(19) United States
(12) Patent Application Publication
(10) Pub. No.: US 2011/0245650 A1
(43) Pub. Date: Oct. 6, 2011

(54) Method and System for Plaque Lesion Characterization

(76) Inventors: William S. Kerwin, Seattle, WA (US); Hui Hu, Seattle, WA (US); Dongxiang Xu, Seattle, WA (US); Michael George Hartmann, Topsfield, MA (US)

(21) Appl. No.: 12/753,502

(22) Filed: Apr. 2, 2010

(51) Int. Cl. A61B 5/05; G06K 9/62

(52) U.S. Cl. 600/407; 382/128

(57) ABSTRACT

A method and system for in-vivo characterization of lesion feature is disclosed. Using a non-invasive medical imaging apparatus, an image of an interior region of a patient's body is obtained. The interior region may include lesion feature (such as plaques) components from a list of components. The lesion feature components are identified by classifying each point in the image as either corresponding to one of the lesion feature components in the list of components or not, using image intensity information and image morphology information, a first relationship (such as an intensity score) correlating image intensity information with the components in the list of components and a second relationship (such as a morphology score) correlating image morphology information with the components in the list of components. Further, a variety of lesion feature characteristics is derived from the result of the classification.

### TECHNICAL FIELD

This disclosure relates generally to methods for assessing a patient's risk associated with atherosclerosis and, more particularly, to clinically efficient methods for characterizing such risks.

### BACKGROUND

Cardiovascular disease resulting from atherosclerosis is a leading cause of mortality and morbidity worldwide. Growing evidence suggests that the decisive factor determining increased risk for atherosclerotic plaque to cause clinical events is plaque composition and morphology rather than the degree of luminal narrowing as measured by angiography.

Atherosclerosis is a form of arteriosclerosis that is characterized by the deposition of plaques containing cholesterol and lipids on the innermost layer of the walls of arteries. The condition usually affects large- and medium-sized arteries. Although such plaque deposits can significantly reduce the blood's flow through an artery, the more serious risk is generally associated with the instigation of an acute clinical event through plaque rupture and thrombosis. In particular, serious damage can occur if an arterial plaque deposit becomes fragile and ruptures, fissures, or ulcerates. Plaque rupture, fissure, or ulcer can cause blood clots to form that block or occlude blood flow and/or break off and travel to other parts of the body. If such blood clots block a blood vessel that feeds the heart, it causes a heart attack; if the blood clot blocks a blood vessel that feeds the brain, it causes a stroke. Similarly, if blood supply to the arms or legs is reduced, it can cause difficulty in walking or light exercise and other collateral damage. Studies indicate that thrombotic complications of atherosclerosis remain the leading cause of morbidity and mortality in Western society.

The presence and extent of plaque build up in an individual's arteries can be detected using a variety of techniques that are well known in the field including, for example, magnetic resonance imaging (“MRI”), computed tomography (“CT”), X-ray angiography, and ultrasound. Various methods have been devised for assessing an individual's risk of a clinically significant event such as a stroke or heart attack related to atherosclerotic deposits in an individual's arteries based on the data obtained by these techniques.

Conventional risk assessment methods have mostly focused on evaluating the effect that the plaque deposit has on the blood flow through the artery. However, it has been recognized that the risk associated with rupture, fissure, or ulceration of plaque may be present even when the plaque deposit does not significantly reduce the flow of blood in an artery. Conversely, large plaque deposits may not correlate to high risk of clinically significant events. Thus, one of the more recently developed methods employs scoring systems that summarize key factors of atherosclerotic plaque vulnerability into a quantitative number that describes the current status of the lesion and is directly linked to risk of causing clinical events and/or rapid progression of the disease.

To perform risk assessment efficiently, it is often useful to employ a system and method capable of providing automated, or semi-automated, recognition and delineation of clinically relevant features, and with a convenient and clinically optimized user interface. The present disclosure relates to, among other things, such systems and methods.

### SUMMARY OF THE DISCLOSURE

The present disclosure relates generally to a system and method for identification and delineation of clinically relevant features, such as necrotic cores and calcification regions. In one configuration, at least two types of information (e.g., intensity and morphology) from in vivo and imaging, such as magnetic resonance imaging (MRI), are used to identify and delineate (segment) clinically relevant features. In one example procedure, each subset (such as a pixel) of the image is first assigned a set scores based on at least two attributes, such as intensity (“intensity score”) and relative position of the subset (“morphology score”); the boundaries delineating each type of relevant feature are automatically calculated based on the scores of the subsets.

A further aspect of the present disclosure relates to assessing the risk of a clinically significant event by multiple assessment methods. In one example, a patient's risk for stroke may be first assessed based on the degree of stenosis of the carotid artery. If the patient is deemed to suffer from severe stenosis, surgical intervention (including, e.g., carotid endarterectomy (“CEA”) and stenting) or other appropriate treatments for reducing or eliminating stroke risks may be indicated; if the stenosis is deemed moderate, a second, more precise method is used to assess the risk. The second method can be, for example, based on the plaque composition, morphology, and/or status.

Another aspect of the present disclosure relates to a computerized method and computer user interface facilitating automatic or semi-automatic assessment of clinically significant events. In one example, the computer is configured to constrain the sequence of the steps in plaque feature characterization and/or risk assessment that the user may take. For example, a mandatory sequence may be the following: (a) selecting MRI image sequences as bases for plaque feature characterization and/or risk assessment; (b) identifying and marking the blood vessel boundaries; (c) aligning (registering) the series of images chosen in (a) with each other; (d) delineating plaque regions; and (e) analysis based on the result of the previous steps.

### BRIEF DESCRIPTION OF THE DRAWINGS

FIG. 1 is a schematic representation of a portion of a typical carotid artery;

FIG. 2 is a schematic sketch of a magnetic resonance image of a transverse cross-section through section 2-2 of the external carotid artery shown in FIG. 1;

FIG. 3 is a schematic diagram of an example plaque feature characterization and/or risk assessment system according one aspect of this disclosure.

FIG. 4 is a schematic illustration of an example configuration of the local computer device 400 in FIG. 3.

FIG. 5 is a flow chart showing an example process for plaque feature characterization and/or risk assessment in one aspect of this disclosure.

FIG. 6(a) is a schematic illustration of one series of slices (solid straight lines) imaged at a particular contrast weighing (e.g., T1-weighted).

FIG. 6(b) is a schematic illustration of a different series of slices (solid straight lines) from those shown in FIG. 6(a) imaged at a different particular contrast weighing (e.g., time-of-flight-weighted). The dashed lines denote the locations of the images calculated by interpolating the image data from the slices marked by the solid lines. At least a subset (A, B and C) of the images in FIG. 6(a) are in longitudinal alignment with at least a subset (D, E and F, respectively) of the interpolated images (dashed lines).

FIG. 7 shows an example saggital image of an external carotid artery near a bifurcation. The superimposed straight line marks the bifurcation 710.

FIGS. 8(a), (b), (c) and (d) are a set of four example MRI images that are simultaneously displayed on a display device of the plaque feature characterization and/or risk assessment system according to one aspect of this disclosure. The four images are longitudinally aligned with each other, all being from the slice at the bifurcation marked in FIG. 7, but have mutually different contrast weighings, respectively.

FIG. 9 shows an example of the deterministic segmentation algorithm applied to phantom images with three contrast weightings (top row) according to one aspect of the disclosure. The intensity score for calcification (left column, upper) shows a bright spot corresponding to the dark region in all contrast weightings. The intensity score for core (left column, lower) shows a bright spot corresponding to the region that is bright on T1W and relatively dark on T2W. After multiplication by the morphology score (middle column), spurious regions near the boundaries are eliminated. See combined images in the right column.

### DETAILED DESCRIPTION

### I. Overview

This disclosure relates generally to efficient feature characterization and/or assessment of a patient's risk for certain clinically significant events based on non-invasive imaging techniques. In one aspect, this disclosure relates to assessment of a patient's risk of suffering a stroke based on multi-contrast-weighing MRI data.

Conventional risk assessment methods have mostly focused on evaluating the effect that the plaque deposit has on the blood flow through the artery. However, studies have established that plaque tissue composition and distribution may strongly influence its clinical course and the likelihood that an atherosclerotic deposit will precipitate a clinical event. For example, a thin fibrous cap covering a large, lipid-rich necrotic core appears to be a clear marker of vulnerable (i.e., high risk) plaque. The “fibrous cap” is a distinct layer of connective tissue that typically covers the lipid core of a plaque deposit. The fibrous cap generally comprises smooth muscle cells in a collagenous-proteoglycan matrix, with varying degrees of infiltration by macrophages and lymphocytes.

A thinning fibrous cap indicates weakened structural integrity and possible future rupture that may lead to an embolic event. In a study of patients using carotid magnetic resonance imaging (“MRI”) to image a portion of the carotid artery prior to undergoing a carotid endarterectomy, the prevalence of fibrous cap rupture, juxtaluminal hemorrhage (thrombus) and juxtaluminal calcification was significantly higher in symptomatic plaque deposits as compared to asymptomatic deposits. Furthermore, in a landmark study based on coronary autopsy specimens, ruptured fibrous cap, calcium nodules, and endothelial erosions were highly correlated with sudden cardiac death. (Virmani et al., Lessons From Sudden Coronary Death: A Comprehensive Morphological Classification Scheme for Atherosclerotic Lesions, Arterioscler. Thromb. Vasc. Biol. 20:1262-1275, 2000.)

In a more recent development, as disclosed in the U.S. Pat. No. 7,340,083 (to Yuan et al.), which is incorporated herein by reference, a scoring system is used to summarize key factors of atherosclerotic plaque vulnerability into a quantitative number that describes the current status of the lesion and is directly linked to risk of causing clinical events and/or rapid progression of the disease. This scoring approach accounts for juxtaluminal characteristics of atherosclerotic plaque including the status of the fibrous cap and the presence of any or all main plaque tissue components such as hemorrhage, lipid rich necrotic core, and calcification, as well as inflammatory activity, and their relative distance to the vessel lumen. This plaque information is non-invasively acquired in vivo, for example, using MRI. A primary application of the atherosclerotic risk scoring can be found in the clinical diagnosis of human carotid atherosclerosis.

In one example, one or more cross-sectional images of an artery are taken, for example, by magnetic resonance imaging, computed tomography, ultrasonics, positron emission tomography, or the like, including possibly using combinations of one or more of these imaging modalities. Components of the plaque—such as necrotic core, hemorrhage, and calcification—are identified and located relative to the juxtaluminal region of the artery. The image is also analyzed to determine the status and composition of the fibrous cap. For example, the fibrous cap may be collagen or mixed tissue (sometimes referred to as “loose matrix”) and may be intact or ruptured. An atherosclerotic risk score is then calculated that characterizes the risk associated with the imaged portion of the artery that is dependent on the fibrous cap status and composition and the present of the identified components in the juxtaluminal region of the artery.

Further examples of risk assessment based on the characteristics of plaque components and/or status and composition of the fibrous cap can be found in the U.S. patent application Ser. Nos. 11/445,510 (filed on 1 Jun. 2006), 11/690,063 (filed on 22 Mar. 2007), and U.S. Provisional Application Nos. 61/184,700, all of which are incorporated herein by reference.

The present disclosure describes alternative and/or supplemental methods and systems to those known in the existing art. The methods and systems disclosed in the present disclosure provide their unique advantages and minimize and avoid certain shortcomings associated with the methods and systems of the existing art. In one aspect, a deterministic method can be used for delineating plaque components, such as necrotic cores and regions of calcification. In another aspect, a computerized system and method provide a user interface (“UI”) that guides the user through a predetermined sequence of steps to complete the analysis of data to arrive at a conclusion (which can be a numerical score) about the level of the patient's risk for certain clinically significant events. Further aspects of the present disclosure are evident in the remainder of the disclosure.

### II. Example Regions-of-Interest

The present disclosure describes methods and systems for plaque feature characterization and/or risk assessment based on image data obtained from certain regions-of-interest (“ROIs”). In one example, image data, such as MRI data, are obtained from the carotid artery and analyzed to assess the patient's risk for stroke.

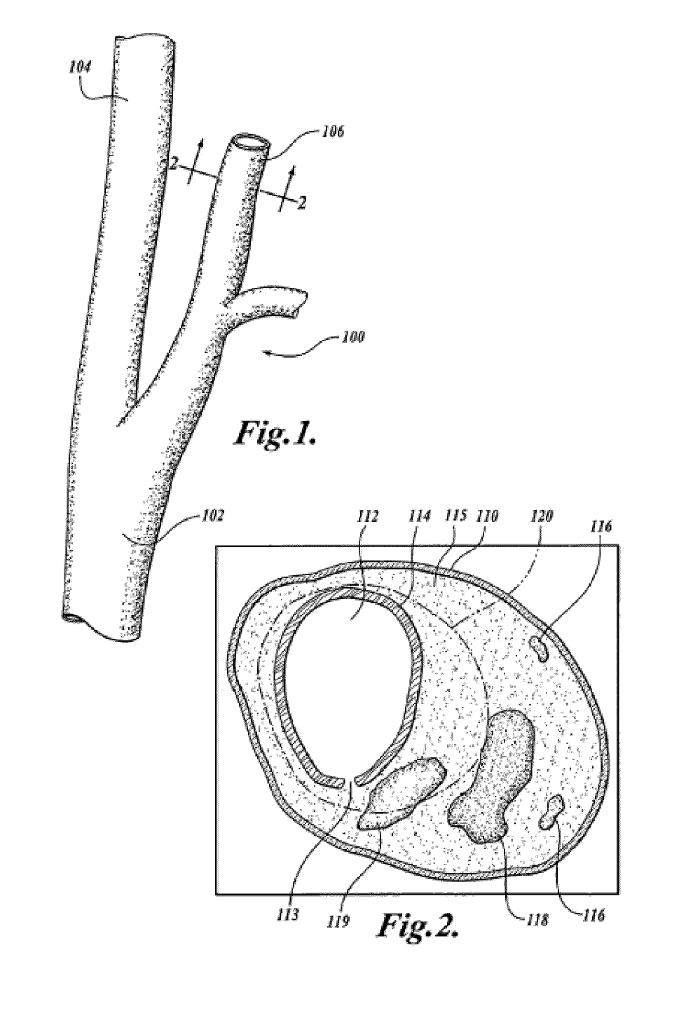

Refer to FIG. 1, which schematically shows a portion of a carotid artery 100 showing the bifurcation of the common carotid artery 102 into the internal carotid artery 104 and the external carotid artery 106. FIG. 2 schematically shows an exemplary MRI image taken through a cross-section of the external carotid artery 106 at section 2-2 of FIG. 1. FIG. 2 is a simplified depiction of a high-resolution MRI image, presented here to facilitate understanding of the present invention. In practice, a clinician or other healthcare professional may examine more than one image to identify specific features of the atherosclerotic deposit. For example, a skilled clinician can identify in the MRI image(s) the artery 106, outer wall 110, the atherosclerotic plaque 115 therein, and other components of the plaque 115, as discussed below. Alternatively, a computer running image analysis software may be used to identify or facilitate identification of these components.

In the exemplary image shown in FIG. 2, the atherosclerotic plaque 115 is substantial. A lumen 112 provides a flow path for the blood and a relatively narrow fibrous cap 114 forms the interface between the lumen 112 blood flow and the rest of the plaque deposit 115. The fibrous cap 114 may be ruptured, as indicated at 113, which may appear in the MRI image as a light or a dark area on the fibrous cap 114. The plaque 115 may include one or more regions of calcification 116 (two shown), one or more necrotic core region(s) 118 and/or hemorrhage(s) 119.

The location of early or recent hemorrhage 119, necrotic core 118, and calcification 116 can also be identified from the MRI image(s)—in particular, the radial position with respect to the lumen 112. In certain applications, a juxtaluminal region can be identified, as indicated by the dotted line 120, to determine if these components are partially or wholly within the juxtaluminal portion of the plaque deposit 115.

### III. Example Processes and Configurations

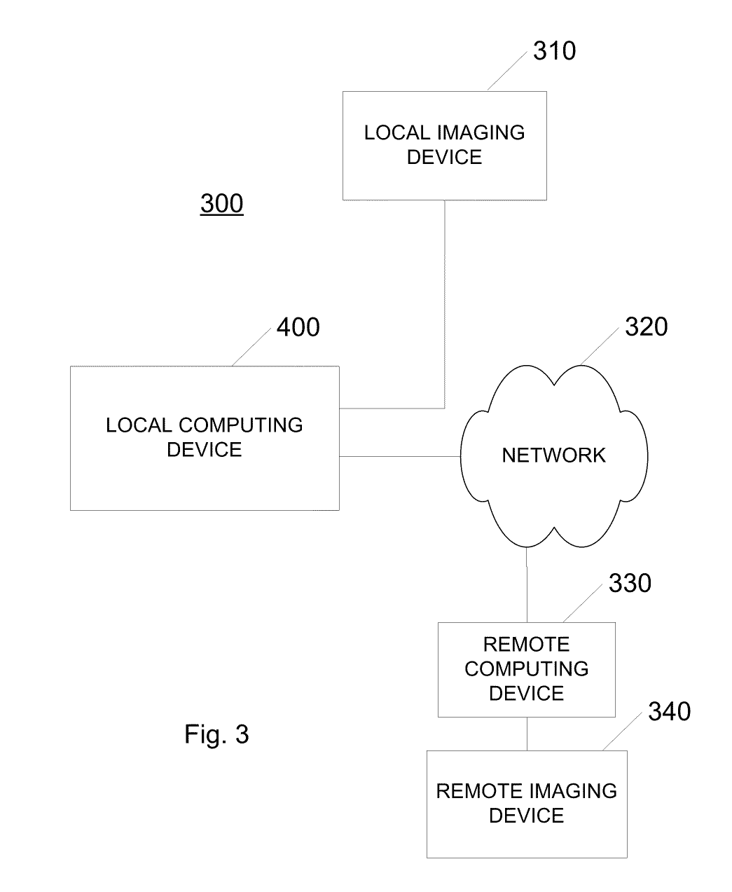

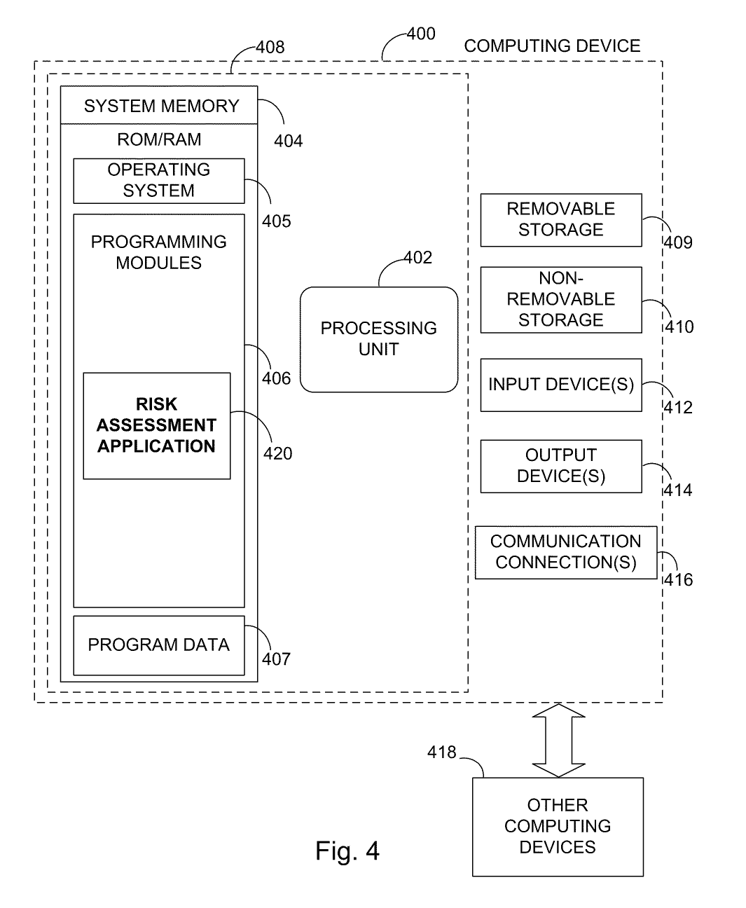

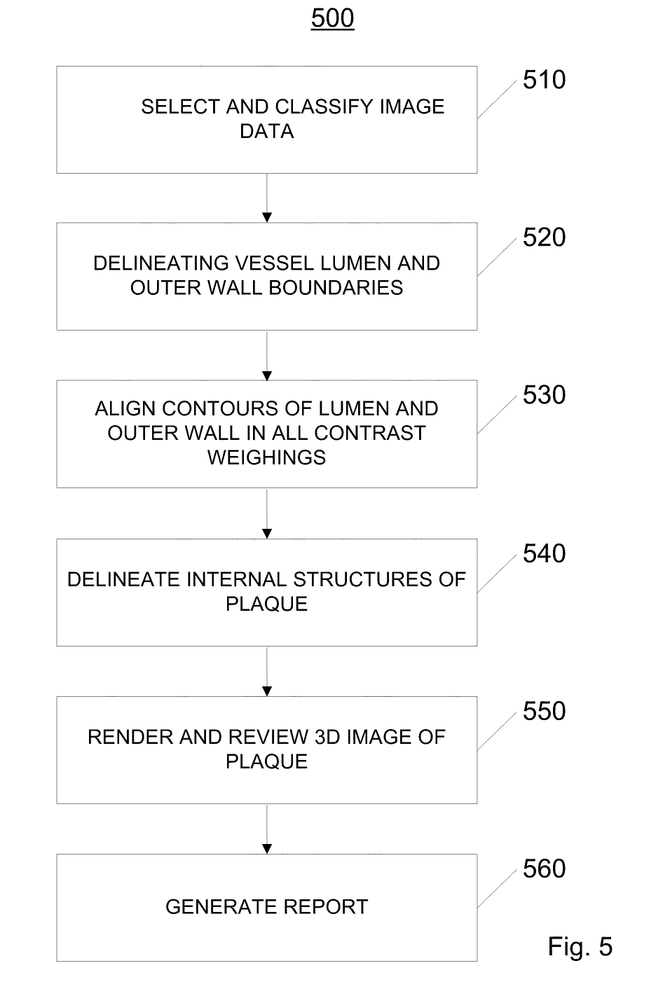

An example of a computer-aided process for characterizing plaque feature and/or assessing a patient's risk associated with atherosclerosis is now described with reference to an example system schematically illustrated in FIGS. 3 and 4 and other illustrative aspects depicted in FIGS. 5-9.

A. Example of Feature Characterization and/or Risk Assessment System

As schematically shown in FIG. 3, a feature characterization and/or risk assessment system 300 according to one aspect of this disclosure includes a local computing device 400, to be described in more detail with further reference to FIG. 4. The computing device 400 can be operatively connected to other electronic devices, such as a local imaging device (e.g. MRI scanner, computed tomographic (“CT”) scanner, ultrasound scanner, positron emission tomography (“PET”) scanner, and the like). The local computing device 400 can also be operatively connected to one or more remote electronic devices via a network 320. The remote electronic devices can include, for example, a remote computing device 330, which, in turn, can operate a remote imaging device.

An “imaging device”, as used in this disclosure, is any device capable of generating signals susceptible to being processed to produce position-dependent data, whether the device itself produces actual visual images.

With reference to FIG. 4, an example computing device 400, in one configuration, includes at least one processing unit 402 and a system memory 404. Depending on the configuration and type of computing device, system memory 404 may comprise, but is not limited to, volatile (e.g. random access memory (RAM)), non-volatile (e.g. read-only memory (ROM)), flash memory, or any combination. System memory 404 may include operating system 405 suitable for controlling computing device 400's operation, one or more programming modules 406, and may include a program data 407. In one aspect, programming modules 406 can include, for example, feature characterization and/or risk assessment application, also called analysis application 420. Thus, whether the computing device 400 is otherwise a general-purpose computer or specifically designed to run the plaque feature characterization and/or risk assessment application, the computing device 400 become configured as a special-purpose computing device for feature characterization and/or risk assessment when the plaque feature characterization and/or risk assessment application 420 is the active application. Furthermore, example processes of this disclosure can be practiced in conjunction with a graphics library, other operating systems, or any other application program and is not limited to any particular application or system. This basic configuration is illustrated in FIG. 4 by those components within a dashed line 408.

Computing device 400 can have additional features or functionality. For example, computing device 400 may also include additional data storage devices (removable and/or non-removable) such as, for example, magnetic disks, optical disks, or tape. Such additional storage is illustrated in FIG. 4 by a removable storage 409 and a non-removable storage 410. Computer storage media may include volatile and nonvolatile, removable and non-removable media implemented in any method or technology for storage of information, such as computer readable instructions, data structures, program modules, or other data. System memory 404, removable storage 409, and non-removable storage 410 are all computer storage media examples (i.e. memory storage.) Computer storage media can include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 400. Any such computer storage media may be part of device 400. Computing device 400 may also have input device(s) 412 such as a keyboard, a mouse, a pen, a sound input device, a touch input device, etc. Output device(s) 414 such as a display, speakers, a printer, etc. may also be included. The aforementioned devices are examples and others can be used.

Computing device 400 can also contain a communication connection 416 that allow device 400 to communicate with other computing devices 418, such as over a network (e.g. network 320) in a distributed computing environment, for example, an intranet or the Internet. Communication connection 416 is one example of communication media. Communication media can typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media can include wired media such as a wired network or direct-wired connection, and wireless media such as acoustic, radio frequency (RF), infrared, and other wireless media. The term computer readable media as used herein may include both storage media and communication media.

As stated above, a number of program modules and data files can be stored in system memory 404, including operating system 405. While executing on processing unit 402, programming modules 406 (e.g., plaque feature characterization and/or risk assessment application 420) can perform processes including, for example, one or more of the steps of risk assessment, as described below. Other programming modules that can be used in accordance with aspects of this disclosure can include word processing applications, spreadsheet applications, database applications, slide presentation applications, drawing or other computer-aided application programs, etc.

In one aspect of this disclosure, the plaque feature characterization and/or risk assessment application 420 includes an image analysis software toolset that facilitates quantitative analysis of blood vessel MRI data sets through semi-automatic or manual contouring and labeling of structures within a user-selected region of interest. In one example, the plaque feature characterization and/or risk assessment application 420 includes a user interface designed to follow prescribed clinical workflow patterns to process, review, validate/edit and analyze digital images.

The image data which the plaque feature characterization and/or risk assessment application 420 acts upon can be in any suitable format. For example, the image data can include one or more MRI series in the Digital Imaging and Communications in Medicine (“DICOM”) format. The image data can be accessed at any suitable location, including a memory in the computing device 400 itself or one or more of the electronic devices operatively connected to the computing system 400.

B. Example Process

With reference to FIGS. 5, 6, 7 and 8, in a process 500 to analyze the image data to assess a patient's risk for a clinically significant event such as stroke, the user first selects a specific patient and exam for analysis (510). In this step, DICOM headers of the images are read to determine which images meet the analysis requirements (based on MRI scan parameters set at the time of imaging). The images that meet the requirements are classified according to patient, date of exam, and contrast weighting (e.g. T1-weighted (“T1W”), T2-weighted (“T2W”), time-of-flight weighted (“TOF”), and/or proton density weighted (“PDW”)).

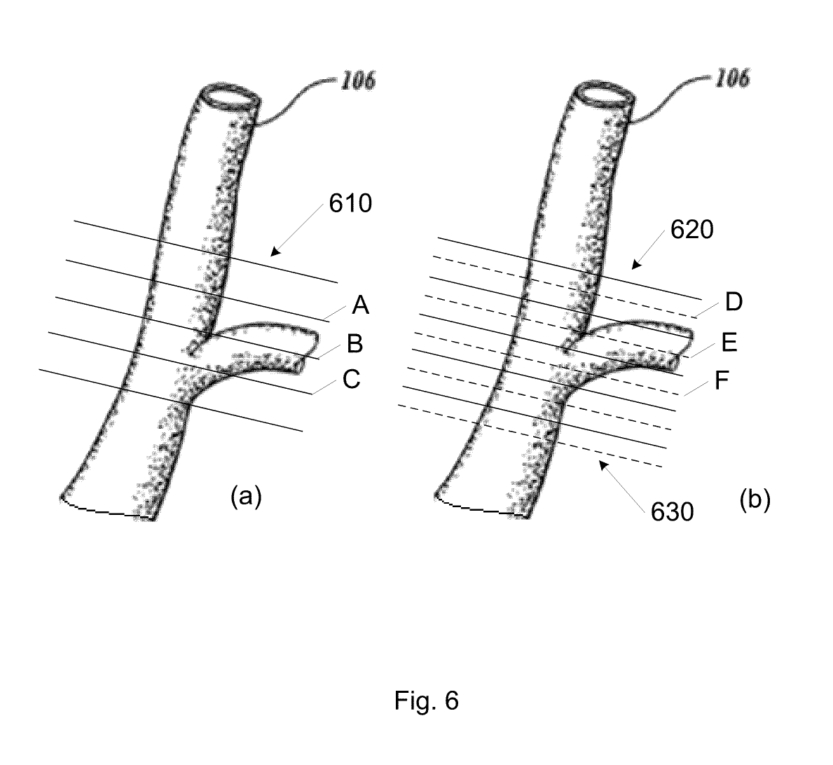

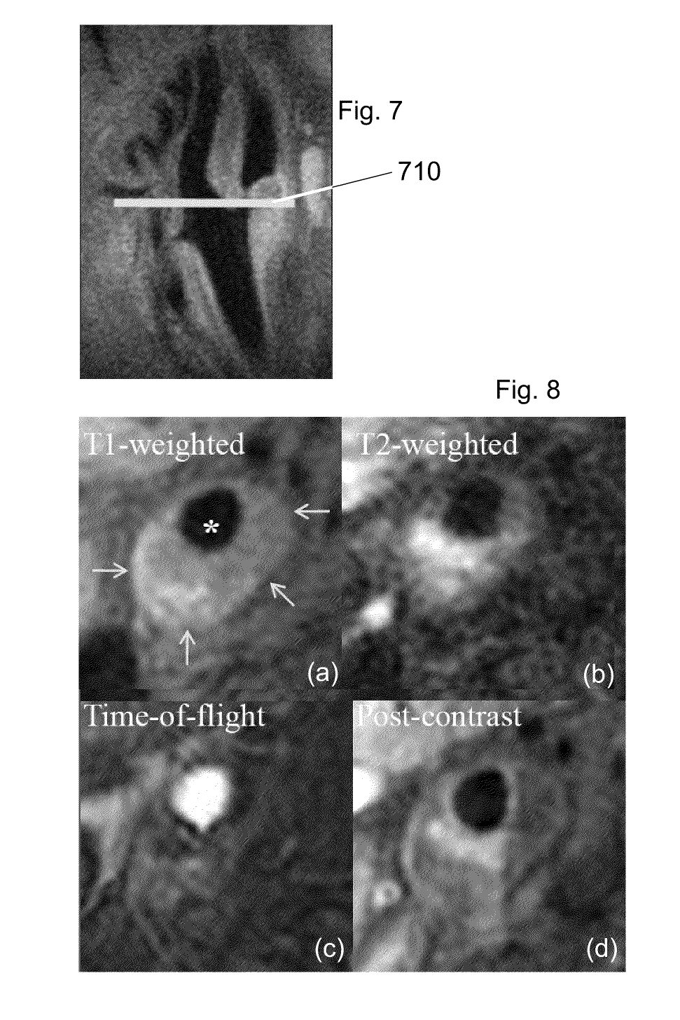

The user then selects which contrast weightings are to be included in the analysis, sets the longitudinal extent (number of slices) of the analysis, and establishes the longitudinal alignment of the images by selecting the location of a common landmark (e.g. the carotid artery bifurcation (e.g., B in FIGS. 6(a) and 710 in FIG. 7)) in all series and locks all series. In one specific example, multiple images, one from each contrast weighing series, are simultaneously displayed on a display device, such as a computer monitor, as shown in FIG. 8. Each series is shifted in the longitudinal direction as needed until the image at a common landmark is displayed. At this point, the user can issue a command (e.g., by clicking on a button in the graphical user interface of the plaque feature characterization and/or risk assessment application 420) to cause two or more of the series to shift longitudinally in synchronization with each other when one of the series is moved.

In one example, after the locking, changing the vertical location of displayed image in one series will cause all series change in sync. In another example, the user can designate one of the series (e.g., the T1W series, FIG. 8(a)) is as the primary series. The plaque feature characterization and/or risk assessment application 420 is configured such that before the locking, changing the vertical location of displayed image in the primary series will cause all series change in sync as if locked, while changing the vertical location of displayed image in a non-primary series will not cause the other series to change in display; after locking, moving any one of the displayed images will cause the rest of the images to change in sync.

In another aspect of this disclosure, two or more image series having no common image plane can be used together. For example, as schematically illustrated in FIGS. 6(a) and (b), the series 610 (solid lines in FIG. 6(a)) in a first contrast weighing (e.g., T1W) has a slice (B) through the bifurcation; the series 620 (solid lines in FIG. 6(b)) in a second contrast weighing (e.g., TOF) does not have any slice passing through the bifurcation. Is such a case, calculations from the image data of the second series can be carried out by the plaque feature characterization and/or risk assessment application 420 to generate a set of interpolated images 630 (dashed lines in FIG. 6(b)) such that at least a subset (D, E and F) of the interpolated images of the second contrast weighing can be longitudinally aligned with a subset (A, B and C, respectively) of the images of the first series. It is further understood that two series with different slice spacing or orientations, or both, can be longitudinally aligned by, for example, generating interpolated images. For example, interpolated slices can be generated from the three-dimensional image data, which is the combined two-dimensional image data from two or more slices.

Once the user has selected a region of interest (ROI) for analysis, the user is guided to the next activity in analysis with the plaque feature characterization and/or risk assessment application 420: Delineating the vessel lumen and outer wall boundaries in each serial, cross-sectional slice (520). Delineation of these boundaries can be accomplished either with manual drawing tools or with semi-automatic boundary delineation tools, as described in more detail below, using the plaque feature characterization and/or risk assessment application 420. Either method permits manual editing of the results.

In one example, the user can delineate the lumen and outer wall boundaries of the vessel in each cross-sectional location for one chosen contrast weighting (the primary series). The user may identify the lumen boundary either by placing a seed point (“*” in FIG. 8(a)) inside the lumen or by placing a set of at least 4 seed points along the lumen boundary. In either case, boundary delineation algorithms automatically delineate the optimal closed contour corresponding to the lumen. In this example, a user input, i.e., locations of the seed or seeds, in addition to the image data, is used by the algorithm to calculate the boundary delineate the feature of interest. An example boundary delineation algorithm is described in Paragios N., Deriche R. “Coupled Geodesic Active regions for Image Segmentation: a Level Set Approach. ECCV. 2000; 224-240, which is incorporated herein by reference. The user reviews the result and can manually adjust the lumen boundary, for example, by clicking and dragging a portion of the automatically calculated contour using a pointing device such as a mouse or via a touch-screen user interface. A lumen boundary identified at one location may also be used to identify lumen boundaries at adjacent locations.

Once the lumen boundary has been identified, the user delineates the outer wall boundary in the same contrast weighting using either a semi-automated delineation algorithm or by placing at least 4 seed points along the boundary (arrows in FIG. 8(a)). In either case, boundary delineation algorithms automatically delineate the optimal closed contour corresponding to the outer wall. The user may review the result and manually adjust this result. A wall boundary identified at one location may also be used to identify lumen boundaries at adjacent locations.

In an optional step 530, once the wall and lumen boundaries are established, an automatic algorithm for image registration automatically aligns the contours drawn on one contrast weighting with features visible in all other contrast weightings in the analysis. The results are reviewed by the user and remaining misalignments are addressed either by manual adjustment of the misalignment and/or manual adjustment of the contours to better match the image features.

In another optional step 540, the user can also delineate and label the internal structures of the vessel wall (between the lumen and outer wall contours) using either manual drawing and labeling techniques or semi-automatic contours delineation and labeling algorithms. One example of such a semi-automatic plaque contours delineation algorithm is disclosed in U.S. patent application Ser. No. 11/445,510, filed on Jun. 1, 2006, and published as U.S. Patent Application Publication No. 2008/0009702 A1, which application is incorporated herein by reference. In another example, a delineation algorithm (see below) automatically delineates regions consisting of calcified and soft (non-calcified) plaque. Manual drawing of calcified and soft plaque regions can also be performed.

In general, manual drawing can be used to delineate other region types. The resulting contours are fully editable by the user as well. The user may review and manually edit, delete, or add to the contours obtained from automated delineation.

In addition, the software can also highlight the region between soft plaque (or lipid-rich necrotic core) contours and the lumen contour and provide area and thickness measurements of this region, referred to as the fibrous cap or the cap.

In another optional step 550, the user can view rendered images (for example, using maximum intensity projection reformat) of the MR images and three-dimensional renderings of the delineated regions. These rendering methods are standard in the industry.

In a final analysis step 560, the plaque feature characterization and/or risk assessment application 420 generates one or more reports, which can include the information, either in summary or for each location, derived from the user generated contours. Such information can include one or more of the following:
Length of artery segment,
Total wall area,
Maximum wall thickness,
Total volumes of all identified regions, by type,
User specified cross-sectional or rendering results,
Stenosis measurements,
Cross-sectional area of each identified contour,
Mean, maximum, and minimum thicknesses of artery wall at the location, and
Images from all contrast weightings with identified contours.

The report can be saved in any suitable format, including PDF, CSV, DICOM, and XML file formats.

At any point, the analysis results may be saved to a file that can be reloaded (restored) for further editing or review.

Workflow Management

Analyzing atherosclerotic plaque consists of multiple complex procedures and requires training. To ensure the average user can consistently obtain a high quality result, a Workflow enforced process, such as the one discussed above, is used to force the user to conduct analysis in an optimal sequence pre-designed by experienced users.

A principal feature of the software will be a streamlined user interface that guides the users through a set sequence of intuitive steps to complete the analysis. Each step will permit only specified activities to be performed. At the end of each step, a validation check will be made to ensure that all analysis steps meet pre-specified constraints. To proceed from the process 510, the user must specify at least one series for analysis, corresponding images must exist for all chosen series, and a landmark location must be specified and series must be locked.

One embodiment for validating process in 520, 530, 540 is that before proceeding to next step, one lumen contour and one wall contour must exist for each location, the lumen contour must be wholly contained within the wall contour, all other contours must be contained between the wall contour and the lumen contour

At any point in the analysis, the user is able to save the results and capture the workflow status in a file, which can be restored at a later time. The analysis can be continued or modified.

C. Detailed Description of Example Automated Algorithms.

In one aspect of this disclosure, the plaque feature characterization and/or risk assessment application 420 provides a set of automated algorithms to assist the user in completing the analysis. A description of an example of each of the algorithms is set forth below.

In certain examples, the plaque feature characterization and/or risk assessment application 420 utilizes one or more of the following automated algorithms:
Propagating active-contour-based delineation of the lumen boundary (Lumen Snake in Vessel Delineation),
Propagating active-contour-based delineation of the outer wall boundary (Outer Wall Snake in Vessel Delineation),
In-plane shifting of images to align with boundaries (Registration),
Division of wall region into sub-regions based on similar image intensity information (Plaque Delineation), and
Computation of thickness between two contours (Thickness Map).

Aspects of these algorithms are based on well-established mathematical models. All algorithms use analysis of grayscale and morphological information.

The general principles of each algorithm are described below. Detailed principle of these algorithms can be found in the following references, all of which are incorporated herein by reference:
Brigger P, Hoeg J, Unser M. B-spline snakes: a flexible tool for parametric contour detection. IEEE Trans Image Proc. 2000; 9:1484-1496.
Cai J, Hatsukami T S, Ferguson M S, et al. In vivo quantitative measurement of intact fibrous cap and lipid-rich necrotic core size in atherosclerotic carotid plaque: comparison of high-resolution, contrast-enhanced magnetic resonance imaging and histology. Circulation. 2005; 112:3437-44.
Chu B., Kampschulte A., Ferguson M. S., et al. Hemorrhage in the atherosclerotic carotid plaque: A high-resolution MRI study. Stroke. 2004; 35:1079-84
Fukunaga K, Hostetler L D. The estimation of the gradient of a density function with applications in pattern recognition. IEEE Trans. Inf. Theory. 1975; 21:32-40.
Kass M., Witkin A. and Terzopoulos D. Snakes: active contour models I J Comput Vis. 1987; 1:321-31.
Kerwin W., Xu D., Liu F, et al. Magnetic resonance imaging of carotid atherosclerosis: plaque analysis. Top Magn Reson Imaging. 2007; 18:371-8.
Liu F., Xu D., Ferguson M. S., et al. Automated in vivo Segmentation of Carotid Plaque MRI with Morphology-Enhanced Probability Maps. Magn Reson Med. 2006; 55:659-668
Mitsumori L. M., Hatsukami T. S., Ferguson M. S., et al. In vivo accuracy of multisequence MR imaging for identifying unstable fibrous caps in advanced human carotid plaques. J Magn Reson Imaging. 2003; 17:410-20
Moody A R, Murphy R E, Morgan P S, et al. Characterization of complicated carotid plaque with magnetic resonance direct thrombus imaging in patients with cerebral ischemia. Circulation. 2003; 107:3047-52.
Murphy R E, Moody A R, Morgan P S, et al. Prevalence of complicated carotid atheroma as detected by magnetic resonance direct thrombus imaging in patients with suspected carotid artery stenosis and previous acute cerebral ischemia. Circulation. 2003; 107:3053-8.
Paragios N., Deriche R. Coupled geodesic active regions for image segmentation: a level set approach. ECCV. 2000; 224-240.
Saam T, Ferguson M S, Yarnykh V L, et al. Quantitative evaluation of carotid plaque composition by in vivo MRI. Arterioscler Thromb Vasc Biol 2005; 25:234-239.
Saam T, Cai J M, Cai Y Q, et al. Carotid plaque composition differs between ethno-racial groups: an MRI pilot study comparing mainland Chinese and American Caucasian patients. Arterioscler Thromb Vasc Biol 2005b; 25:611-6.
Saam T, Cai J, Ma L, et al. Comparison of symptomatic and asymptomatic atherosclerotic carotid plaque features with in vivo MR imaging. Radiology. 2006; 240:464-72
Shinnar M., Fallon J. T., Wehrli S., et al. The diagnostic accuracy of ex vivo MRI for human atherosclerotic plaque characterization. Arterioscler Thromb Vasc Biol. 1999:19, 2756-61.
Takaya N, Yuan C, Chu B, et al. Association between carotid plaque characteristics and subsequent ischemic cerebrovascular events: a prospective assessment with MRI—initial results. Stroke. 2006; 37:818-23.
Trivedi R A, U-King-Im J M, Graves M J, et al. MRI-derived measurements of fibrous-cap and lipid-core thickness: the potential for identifying vulnerable carotid plaques in vivo. Neuroradiology. 2004; 46:738-43.
Yuan C., Mitsumori L. M., Ferguson M. S., et al. In vivo accuracy of multispectral magnetic resonance imaging for identifying lipid-rich necrotic cores and intraplaque hemorrhage in advanced human carotid plaques. Circulation. 2001; 104:2051-6

i. Lumen Contour Delineation Algorithms

The plaque feature characterization and/or risk assessment application 420, in one example, uses B-splines to define the lumen boundary (Kerwin 2007). B-splines are widely used to define closed curves (for example in Microsoft Powerpoint). The resulting contours can be easily modified by manually dragging the control points of the B-spline.

B-spline snake: To automatically optimize a lumen contour, plaque feature characterization and/or risk assessment application 420 uses active contour (“snake”) techniques (Kass 1987), which are, a common boundary detection techniques in the industry. In a more specific example, the plaque feature characterization and/or risk assessment application 420 specifically uses a type of B-spline snake described in (Brigger, 2000). The snake seeks to minimize an “energy” function, where the energy is high when the contour is not aligned with a boundary and low when it is aligned. The plaque feature characterization and/or risk assessment application 420's snake begins with a series of initial control points (for example from manual input) that define an initial contour, with an associated energy. The final contour is obtained by modifying the control points using gradient descent until a minimum energy is reached.

Mean-shift segmentation: In addition to manually identifying the initial control points, the plaque feature characterization and/or risk assessment application 420 can also automatically generate initial control points based on a single click of the mouse within the lumen. This is done using a standard “region growing” approach to identify a region with similar intensity to the selected point. One example region growing approach is the “mean shift,” as described in Fukunaga (1975). This process iteratively identifies all points that share a common mean intensity. The boundary of this region is used to initialize the B-spline snake.

Auto-propagation: The plaque feature characterization and/or risk assessment application 420 also features the ability to automatically use a lumen contour from a prior image in finding the next. This is done simply by taking the central point of the prior lumen contour and using it in the mean-shift algorithm described above.

User interaction: This approach allows two mechanisms for rapid user adjustment of the results. First, a threshold in the mean shift segmentation can be adjusted to make the range of values accepted within a common mean lower or higher. Second, the B-spline snake result can be quickly adjusted by moving the control points in the B-spline.

ii. Wall Delineation Algorithms

In one example, the outer wall boundary is delineated using the same B-spline snake as described above for the lumen contour. As in lumen delineation, the wall delineation algorithm can be initialized by user input of control points.

Lumen Expansion: In one aspect of this disclosure, if a user chooses not to enter control points to generate a contour, an automated algorithm can be used to initialize the B-spline contour for the wall. This algorithm cannot rely on mean shift segmentation (as for the lumen) because the outer wall boundary can have diverse brightness levels depending on its makeup. Therefore, the plaque feature characterization and/or risk assessment application 420 uses an approach based on expanding the lumen contour outward. Using a series of increasing outward expansions, the lumen is expanded and then mapped to the closest ellipse. Each ellipse is used to initialize a B-spline snake and the one that produces the overall minimum energy is selected. If the prior location has an outer wall contour, the amount of expansion is proportional to the local thickness on the previous location using a conditional shape model, which is described in, e.g., U.S. patent application Ser. No. 11/690,063, filed Mar. 22, 2007 and published as U.S. Patent Application Publication 2007/0269086 A1, which application is incorporated herein by reference.

User interaction: This approach to vessel wall delineation allows two mechanisms for rapid user adjustment of the results. First, the B-spline snake result can be quickly adjusted by moving the control points in the B-spline as in the lumen contour. Second, the proportionality constant for thickness can be altered from the optimal value by the user. This will yield alternate contour solutions.

iii. Registration Algorithm

To align images obtained with different contrast weightings and at different times in the acquisition process, the plaque feature characterization and/or risk assessment application 420 can automatically compute an in-plane shift in one example (Kerwin 2007). The shift is determined by a search over all possible shifts (within a user-specified limit) that find the one that best aligns the existing lumen and outer wall contours with the features in the image. The optimal shift is determined as the one that minimizes an energy function proportional to the total gradient of the image intensity beneath the lumen and wall contours (i.e., the line integral of the image gradient). This function is minimized when the contours overly edges apparent within the images.

User interaction: In one example, the plaque feature characterization and/or risk assessment application 420 can also provide a user-assisted method within this same framework in which the user drags the image to obtain a rough alignment of the contours with the features. Then the plaque feature characterization and/or risk assessment application 420 identifies the optimal shift within a small window around this point using the algorithm described above.

iv. Plaque Delineation Algorithm

Deterministic algorithm: U.S. patent application Ser. No. 11/445,510 discloses an algorithm of automated in vivo segmentation of atherosclerotic plaque MRI with morphology-enhanced probability maps. This is a statistical based analysis method, where the statistical modeling is captured by the so called probability maps. The probability maps are not a priori knowledge, and therefore have to be developed from a set of statistical training data, the data whose outcomes (analysis results) are known. Typically, statistical training data are obtained from subjects having certain characteristic that are expected to be similar to the characteristics to be ascertained from the patients. Based on the training data, the probability maps are derived by best fitting the outcomes of training data.

In certain situations, it is desirable from the implementation perspective to develop an alternative method that is deterministic rather-than statistical.

Morphology-enhanced segmentation algorithm for plaque delineation is a general-purpose segmentation algorithm that is based on a simple mathematical model. This algorithm is tailored for plaque delineation by customizing a few parameters of the algorithm based on accepted practices in the medical literature and performance testing on several cases of vessel wall MRI.

The general approach of the segmentation algorithm is to assign a “score” to each pixel in the image that indicates how well the pixel matches pre-specified characteristics in terms of intensity and location of the pixel. A high score indicates that the pixel closely matches the characteristics and a low score indicates that the pixel does not match.

To assign a score to a pixel, the difference of its intensity from a desired intensity is computed. To account for differences in imaging and hardware configurations that affect absolute MRI intensities, the difference is computed by normalizing to the local median intensity. Also, because multiple contrast weightings are used, the total difference for a given pixel is computed as the root-mean-square of all the individual differences. Then, a score is assigned based on the following plot:

In this plot, the height (h) is the maximum score, and the width (w) is the maximum difference, beyond which the score is 0. This is similar to thresholding except the threshold is “soft” rather than “hard.” In traditional thresholding, the curve would be a step function.

In addition to an intensity factor in the score, a morphology score is also used to provide a “buffer” zone near the lumen and wall contours, where plaque components are unlikely to be found. This factor is determined by the minimum of the distance from the pixel to the lumen and wall boundaries according to the following chart:

This factor is multiplied by the intensity score to compute the final score for each pixel. Below the distance threshold (D), the overall score is reduced, whereas above D, the overall score is the same as the intensity score.

The basic segmentation framework of the plaque feature characterization and/or risk assessment application 420 allows up to four sets of intensity and location characteristics to be specified with corresponding labels, essentially generating four scores for each pixel. However, the default configuration only uses two sets of pre-specified characteristics: one for calcified plaque (CA) and one for soft (non-calcified) plaque (SP).

Default Configuration: In one illustrative example, the plaque feature characterization and/or risk assessment application 420 is configured to give results that are consistent with the well-validated findings in the relevant medical literature. For example, a number of papers (Saam 2005; Cai 2005; Trivedi 2004; Mitsumori 2003; Moody 2003; Chu 2005; Yuan 2001; Shinnar 1999) have described rules for identifying plaque components according to relative intensity characteristics (hypointense, isointense, or hyperintense) within different contrast weightings (T1W, T2W, PDW, etc.). These techniques have relied on manual delineation of regions that match the indicated intensity characteristics.

Further, calcified plaque has been characterized by absence of signal in MRI due to a lack of hydrogen nuclei and susceptibility effects of the calcified deposits. Studies have shown that calcified plaque can be identified with high sensitivity and specificity as hypointense regions on all of the multi-contrast weighted (multi-spectral) MR images (Saam 2005; Mitsumori 2003; Shinnar 1999). Numerous studies have been published in the literature that study calcified plaque in the carotid artery using these signal characteristics (Saam 2006; Saam 2005b; Takaya 2006).

Soft plaque regions are areas of the plaque wherein the soft, non-calcified components have been deposited. These regions generally consist of lipids, cholesterol, necrotic debris, and blood products (hemorrhage). These components generally lead to shortening of T1 and T2 values and hence isointense to hyperintense appearance on T1-weighted MR images and isointense to hypointense appearance on T2-weighted MR images. Use of these MRI characteristics to identify soft plaque components has been well validated (Yuan 2001; Chu 2005; Trivedi 2004) and has been accepted as classification criteria in the medical literature (Saam 2006; Saam 2005b; Takaya 2006; Murphy 2003).

In one example configuration, to replicate these rules and in a default setting, the plaque feature characterization and/or risk assessment application 420 sets the desired intensity for calcified plaque to equal 0.5 times the median (hypointense) in both T1-weighted and T2-weighted images. The desired intensity for soft plaque is set to equal 1.5 times the median (hyperintense) in T1-weighted images and to 1.0 times the median (isointense) in T2-weighted images. The width of the ramp function for the intensity score (w) is set to equal 1.0 times the median. Finally, the optimal peak values were found to be 21 for calcified plaque and 13 for soft plaque based on testing on a number of test cases. Likewise, the optimal value of D was found to be 1.5 mm, which corresponds to typical normal thicknesses of large vessel walls. These setting may be reconfigured based on intuition preference.

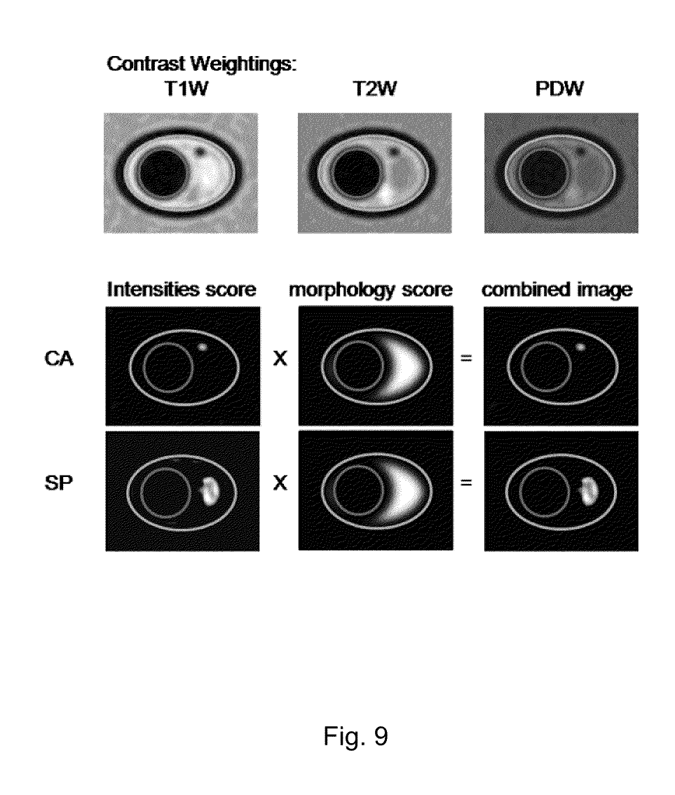

The generation of scores for all of the image pixels in a set of simulated images is shown in FIG. 9. FIG. 9 shows an example of the deterministic segmentation algorithm applied to phantom images with three contrast weightings (top row) according to one aspect of the disclosure. The intensity score for calcification (left column, upper) shows a bright spot corresponding to the dark region in all contrast weightings. The intensity score for core (left column, lower) shows a bright spot corresponding to the region that is bright on T1W and relatively dark on T2W. After multiplication by the morphology score (middle column), spurious regions near the boundaries are eliminated. See combined images in the right column in FIG. 9.

Competing Active Contours: After the scores for each pixel are determined, for ease of editing, it is desirable to delineate the regions of high scores by contours. For this purpose, plaque feature characterization and/or risk assessment application 420 again utilizes a standard snake algorithm. And, to ensure that contoured regions do not overlap, the method of “competing active contours” (Paragios 2000; Liu 2006) is used.

v. Thickness Mapping Algorithm

The algorithm to determine thickness of the region between two contours is described in U.S. Pat. No. 7,353,117, which is incorporated herein by reference, based on the well-know method of Delaunay triangulation (Schumaker, 1987). Every point along one contour is matched to a corresponding point on the other contour. In combination with the lines connecting adjacent points within the contour, the result is a set of triangles with points on the contours as vertices. The set of contours that maximizes the minimum angle over all triangles in the set is defined as optimal. Delaunay triangulation theory states that this set is unique and provides tools for finding the optimum. Once the optimal set of triangles is found, the lengths of lines connecting inner and outer contours are taken to be the local thicknesses.

vi. User Review and Editing

In one example aspect, the algorithms used in the semi-automatic tools described above for delineating the lumen and outer walls and plaque components are designed to perform operations automatically. The plaque feature characterization and/or risk assessment application 420 allows the user choose to use a manual operation at any time and not use the corresponding semi-automated tools.

In another example aspect, the contours generated by the semi-automated tools described above, as well as contours generated manually without using the semi-automated tools, are stored separately, and not embedded in, the source images. The contours can thus be modified or deleted without affecting the original image. This applies even after an editing review session has been saved to a project. Upon re-opening the project, the contours are as the user left them, and can be modified without affecting the original images.

D. Stratified Screening of Patients

A further aspect of the present disclosure relates to assessing the risk of a clinically significant event by multiple levels of risk assessment.

A common technique currently used to assess stroke risk, for example, is stenosis measurement by techniques such as duplex ultrasound imaging, CT angiography (“CTA”), MR angiography (“MRA”) or X-ray angiography. Patients identified as having severe stenosis (for example, 80-99% occlusion) are considered high risk and are candidates for surgical intervention (such as stent implantation or carotid endarterectomy (“CEA”)), whereas those identified as having moderate stenosis (for example, 50%-79%) could be considered intermediate risk and are candidates for drug treatment (such as with cholesterol-lowering drugs), if they don't have stroke related symptom. However, as stated above, depending on the plaque composition and morphology, a patient may be at risk for stroke even though the patient does not have severe stenosis. It therefore can be beneficial to conduct a second screening of the patients with moderate levels of stenosis to identify those at high stroke risk for appropriate intervention such as surgery.

In one aspect of this disclosure, the second screening can be conducted using a scoring method and system such as those disclosed in U.S. Pat. No. 7,340,083 or in U.S. Provisional Patent Application Ser. No. 61/184,700. The system can be a computerized system with a risk assessment application such as disclosed in this disclosure.

In other examples, the aforementioned risk scoring method and system can be used to provide further levels of screening after one of following groups is identified:
1) Asymptomatic group with moderate stenosis measured by ultrasound, CTA, MRA, or X-ray Angiography,
2) Symptomatic group with moderate stenosis measured by ultrasound, CTA, MRA, or X-ray Angiography,
3) Severe stenosis measured by ultrasound, CTA, MRA, or X-Ray Angiography,
4) low risk group identified by ultrasound, and
5) high risk group identified by ultrasound.

Furthermore, it can be beneficial to screen certain age population, such as all persons older than a certain age (such as 65), using both stenosis measurement and the risk scoring system and methods described above.

F. Treatment Planning Using Plaque Characteristics

In a further example, techniques incorporating the plaque characterization method and system described above can be used to carry out at least one of the following:
a) assessing the risk of complication in a surgical intervention for reducing or eliminating the patient's risk of a clinically significant event associates with the lesion feature,
b) planning surgical intervention for reducing or eliminating the patient's risk of a clinically significant event associates with the lesion feature
c) designing drug treatment of the patient for reducing or eliminating the patient's risk of a clinically significant event associates with the lesion feature; and
d) assessing the patient's response to a treatment for reducing or eliminating the patient's risk of a clinically significant event associates with the lesion feature.

### VI. Summary

Thus, according to the present disclosure, a method and system for efficient assessment of a patient's risk for certain clinically significant events have been described. The deterministic method and the computerized system for running the method provide efficient characterization of plaque component, thereby improving the efficiency of risk scoring. The user interface of the computerized system described herein provides efficient representation and analysis of image data, and provides guidance for the user to following an optimized sequence of steps in risk analysis. Furthermore, a combination of traditional risk assessment method and the scoring system and method, whether or not employing the user interface or deterministic delineation algorithm described above, provides added precision of risk prediction in an efficient manner.

The above specification, examples and data provide a complete description of the make and use of the invention. Since many embodiments of the invention can be made without departing from the spirit and scope of the invention, the invention resides in the claims hereinafter appended.

We claim:

1. A method for in-vivo characterization of lesion feature, the method comprising:
using a non-invasive medical imaging apparatus, obtaining from a patient an image of an interior region of a body, wherein the interior region includes lesion feature components from a list of components, the image having intensity information and morphological information;
identifying lesion feature components by classifying each point in the image as either corresponding to one of the lesion feature components in the list of components or not, using the image intensity information and the image morphology information, a first relationship correlating image intensity information with the components in the list of components and a second relationship correlating image morphology information with the components in the list of components; and
deriving, from the result of the classifying step, a set of lesion feature characteristics including one or more of:
(a) lesion type or types,
(b) total volumes of all identified components, by type, and
(c) cross-sectional area of each identified component,

2. The method of claim 1, wherein obtaining the image from the patient comprises:
obtaining a plurality of series of images of an interior region in the patient, the plurality of series of images, each of the series representing a mapping of a quantity over a plurality of pixels, the plurality of pixels corresponding to respective portions of the interior region;
assigning each subset of the plurality of pixels of each image a plurality of scores, each score based at least in part on a respective attribute of the subset of the pixels; and
classifying each portion or portions of the interior region based at least in part on a combination of the plurality of scores for the corresponding subset of pixels.

3. The method of claim 1, wherein
the first relationship correlating image intensity information with the components in the list of components result in an intensity score, based on an image signal intensity of the pixel;
and the second relationship correlating image morphology information with the components in the list of components result in a morphology score, based on the location of the pixels relative to a reference location; and
wherein the classifying step comprises classifying the pixels based at least in part on a combination of the intensity score and morphology score,

4. The method of claim 3, wherein the first and/or second relationships being derived independent from statistical training data.

5. The method of claim 3, wherein the interior region comprises a portion of a an artery wall containing atherosclerotic plaque having an inner boundary defining a vessel lumen, and having an outer wall boundary, wherein the morphology score is based at least in part on the location of the subset of pixels relative to the pixels corresponding to the inner boundary.

6. The method of claim 5, wherein the classifying step further comprises calculating, using a computer programmed with a predetermined algorithm for delineating plaque components based at least in part on the intensity scores and morphology scores of pixels, a contour delineating at least one of the plaque components in at least one of the plurality of displayed image according the algorithm.

7. The method of claim 6, further comprising:
calculating a contour delineating the vessel lumen and outer wall in at least one of the plurality of displayed images;
shifting at least one of the remaining images from the plurality of displayed images to align the vessel structures with the lumen and outer wall boundaries delineated, when the vessel structures are misaligned with the lumen and outer wall boundaries delineated; and
using one or more user inputs to the computer, providing input to the contour delineating algorithm before calculating the contour, or altering the computer calculated contour independent of the algorithm after calculating the contour, or both.

8. The method of claim 1, wherein the interior region comprises a portion of a an artery wall containing atherosclerotic plaque having an inner boundary defining a vessel lumen, and having an outer wall boundary; wherein the deriving step further comprising deriving an additional set characteristics of the interior region, including one or more of:
(d) stenosis measurements,
(e) length of the artery segment,
(f) total wall area,
(g) maximum wall thickness,
(h) mean, maximum, and minimum thicknesses of artery wall at the location, and
(i) images from all series with identified contours.

9. The method of claim 2, further comprising:
simultaneously displaying a plurality of images, one from each series of images, on a display device;
determining whether the plurality of displayed images correspond to substantially the same portions of the interior region, and if not, displaying at least one different image from one of the plurality of series of images;
repeating the process in the preceding step until the plurality of displayed images correspond to substantially the same portions of the interior region; locking the relative positions between the plurality of series of images after the plurality of displayed images correspond to substantially the same portions of the interior region such that displaying a new image from any series of images automatically causes a new image from at least another series of images to be displayed, and vice versa, with the newly displayed images corresponding to substantially the same portions of the interior region; and

10. The method of claim 1, further comprising assessing the patient's risk of a clinically significant event based on at least some of the lesion features characteristics.

11. The method of claim 10, wherein assessing the patient's risk of a clinically significant event further comprises assessing the patient's risk of the clinically significant event based on at least some of the lesion feature characteristics after the patient has been identified as having at least one of high and intermediate risk of the clinically significant event by at least one other risk assessment method that classifies patients' risk of the clinically significant event into high, low and at least one intermediate levels.

12. The method of claim 1, further comprising, based on the derived lesion feature characteristics, performing at least one of:
a) assessing the risk of complication in a surgical intervention for reducing or eliminating the patient's risk of a clinically significant event associates with the lesion feature,
b) planning surgical intervention for reducing or eliminating the patient's risk of a clinically significant event associates with the lesion feature
c) designing drug treatment of the patient for reducing or eliminating the patient's risk of a clinically significant event associates with the lesion feature; and
d) assessing the patient's response to a treatment for reducing or eliminating the patient's risk of a clinically significant event associates with the lesion feature.

13. A computerized system for plaque feature characterization and/or risk assessing a patient's risk associated with a clinically significant event, the system comprising a computing device comprising at least a programming module operating a plaque feature characterization and/or risk assessment application, the system being configured to carry out, when the plaque feature characterization and/or risk assessment application is active, the steps of:
(a) obtaining a plurality of series of images of an interior region in the patient, the plurality of series of images, each of the series representing a mapping of a quantity over a plurality of pixels, the plurality of pixels corresponding to respective portions of the interior region;
(b) assigning each subset of the plurality of pixels of each image a plurality of scores, each score based at least in part on a respective attribute of the subset of the pixels; and
(c) classifying each portion or portions of the interior region based at least in part on a combination of the plurality of scores for the corresponding subset of pixels.

14. The system of claim 13, wherein the plurality of scores comprises:
an intensity score, based on an image signal intensity of the pixel and a first relationship correlating image intensity information with the components in the list of components; and
a morphology score, based on the location of the pixels relative to a reference location and a second relationship correlating image morphology information with the components in the list of components,
wherein the classifying step comprises classifying the pixels based at least in part on a combination of the intensity score and morphology score,
wherein the interior region comprises a portion of a an artery containing atherosclerotic plaque having an inner boundary defining a vessel lumen, and having an outer wall boundary; wherein the morphology score is based at least in part on the location of the subset of pixels relative to the pixels corresponding to the inner boundary; and the classification step comprises classifying the portions of the interior region as belonging to component, or neither, based on the intensity and morphology scores.

15. The system of claim 14, further configured to carry out the steps of:
(d) calculating, using a predetermined algorithm, contours delineating the regions of pixels corresponding to the vessel lumen and outer wall;
(e) calculating, using the predetermined algorithm, a contour delineating a region of pixels corresponding to a plaque component; and
(f) generating analysis relating to the patient plaque characteristics

16. The system of claim 15, further comprising a user interface configured to constrain a user of the system to conduct feature characterization in the sequence of:
(i) selecting image sequences as bases for plaque feature characterization and/or risk assessment;
(ii) identifying and marking the blood vessel boundaries;
(iii) aligning the series of images chosen in (i) with each other;
(iv) delineating plaque regions; and
(v) characterize the plaque components based at least on the result of steps (i)-(v).

17. The system of claim 16, wherein at least one of the first and second relationships is derived independent from statistical training data.

18. The system of claim 15, wherein the user interface is configured to use a user input to the system to provide input to the contour delineating algorithm before calculating the countours, or alter the computer calculated contour independent of the algorithm after calculating the countours, or both.

19. The method of claim 11, wherein the other risk assessment method comprises using a medical diagnostic apparatus to measure the patient's degree of stenosis,
wherein, the patient is classified into a predefined high-, intermediate-, or low-risk group for stroke based at least on the measured degree of stenosis;
further comprising, in the event that the patient is classified in to the high- or intermediate-risk group, re-classifying the patient into one of the predefined risk groups based on at least some of the lesion feature characterizations.

20. A method for treating a patient for a medical condition or for reducing the patient's risk for a clinically significant event, the method comprising:
using a non-invasive medical imaging apparatus, obtaining from a patient an image of an interior region of a body, wherein the interior region includes lesion feature components from a list of components, the image having intensity information and morphological information;
identifying lesion feature components by associating each point in the image one or more of the lesion feature components in the list of components using the image intensity information and the image morphology information, a first relationship correlating image intensity information with the components in the list of components and a second relationship correlating image morphology information with the components in the list of components;
deriving, from the result of the classifying step, a set of feature characteristics including one or more of:
(a) lesion type or types,
(b) total volumes of all identified components, by type,
(c) stenosis measurements,
(d) cross-sectional area of each identified component; and performing at least one of:
1) assessing the risk of complication in a surgical intervention for reducing or eliminating the patient's risk of a clinically significant event associates with the lesion feature,
2) planning surgical intervention for reducing or eliminating the patient's risk of a clinically significant event associates with the lesion feature
3) designing drug treatment of the patient for reducing or eliminating the patient's risk of a clinically significant event associates with the lesion feature; and
4) assessing the patient's response to a treatment for reducing or eliminating the patient's risk of a clinically significant event associates with the lesion feature.
5) assessing the patient's risk of a clinically significant event further comprises assessing the patient's risk of the clinically significant event based on at least some of the lesion feature characteristics after the patient has been identified as having at least one of high and intermediate risk of the clinically significant event by at least one other risk assessment method that classifies patients' risk of the clinically significant event into high, low and at least one intermediate level.
